(19) United States
(12) Patent Application Publication
(10) Pub. No.: US 2006/0094023 A1
(43) Pub. Date: May 4, 2006

(54) Method for isolating nucleic acid by using amino surfactants

(75) Inventors: Tung-Liang Huang, Hsinchu City (TW); Shang-Chi Lin, Rende Township (TW)

Correspondence Address: BACON & THOMAS, PLLC, 625 SLATERS LANE, FOURTH FLOOR, ALEXANDRIA, VA, 22314, US

(73) Assignee: Industrial Technology Research Institute, Hsinchu (TW)

(21) Appl. No.: 10/978,349

(22) Filed: Nov. 2, 2004

(51) Int. Cl. C12Q 1/68; C07H 21/02; C07H 21/04

(52) U.S. Cl. 435/6; 536/25.4

(57) ABSTRACT

A method for isolating nucleic acids by forming an insoluble ionic complex between nucleic acids and a surfactant in a biological sample, consisting of a step of contacting the biological sample with an isolation composition comprising amino surfactants of the formula (I): R₁R₂R₃N(O)ₓ, (I), wherein, R¹ and R₂ each independently is H, C1-C6 alkyl group, C6-C12 aryl group, or C6-C12 aralkyl group; R₃ is C1-C20 alkyl group, C6-C26 aryl group or C6-C26 aralkyl group; and x is an integer of 0 or 1. Moreover, the concentration of the amino surfactants in the composition ranges from 0.001% to 20%.

### BACKGROUND OF THE INVENTION

1. Field of the Invention

The present invention relates to a method for isolating biomaterials from a sample, and more particularly, to a method for isolating nucleic acid from a biological sample.

2. Description of Related Art

Nucleic acids are known to carry genetic information of an organism. Nowadays, nucleic acids also play important roles in the research fields of molecular biology. According to recent research results, it is known that genetic defects or diseases development of a patient can be deduced from the abnormality or special sequences of nucleic acids of that patient by clinical practice. Thus the goal for preventing disease occurrence can be achieved by detecting the abnormality of nucleic acids and taking necessary remedying steps for treatment before the onset of diseases. To achieve effective detection of abnormality or special sequences of the nucleic acids, the isolation of nucleic acids from an organism, as well as the steps for keeping the genetic information intact are the key subjects for related applications.

To avoid contamination from proteins or other organelles, steps of isolating pure nucleic acids accompanied with application of phenol/chloroform are popular in conventional methods. However, the application of phenol/chloroform in steps for isolating nucleic acids from biological materials requires careful operation and skilled technicians to exclude unwanted organelles or materials (e.g. proteins) from nucleic acids. On the other hand, phenol and chloroform are dangerous for operation and highly polluting to the environment, too. In addition, phenol and chloroform are not able to condense nucleic acids directly. Therefore, many chemicals for assisting phenol or chloroform to condense nucleic acids are also demanded. Moreover, phenols are inconvenient to store and to use because they easily oxidize. According to these drawbacks described above, phenol/chloroform is not the best chemical to use for isolating nucleic acids, especially as it is unsuitable for high-throughput isolation of nucleic acids for clinical biomedical applications.

Several investigations about isolation of nucleic acids without phenol/chloroform have been reported. Qiagen Company disclosed a method in PCT patent No. WO03084976 for isolating nucleic acids with ammonia or ammonium to overcome the drawbacks illustrated above. In this method, a solid phase acting as an attaching substrate for nucleic acids to bind with is used. In addition, a chaotropic agent is present at the very first stage of isolation to melt double-stranded oligonucleotides. Moreover, binding efficiency between denatured nucleic acids and the solid phase is enhanced in the presence of the ammonia or primary amine. Accordingly, the contacting of the sample with the nucleic acid binding solid phase is preferred to be conducted in the presence of the ammonium or ammonia at a pH ranging from 8.5 to 9.5.

On the other hand, in U.S. Pat. No. 6,265,168 to Gjerde et al. discloses a method for removing a target DNA fragment having a predetermined base-pair length from a mixture of DNA fragments. The DNA fragment mixture which contains the target DNA fragment is in a first solvent mixture with a counterion and a DNA binding concentration of driving solvent and is then applied to a separation column containing media having a nonpolar, nonporous surface. The target DNA fragments are separated from the media by contacting the surface with a second solvent solution. The counterion used here are primary amines, secondary amines, lower tertiary amines and quaternary alkylammonium salts. The key factors of this method in U.S. Pat. No. 6,265,168 for purifying a target DNA fragment are the column with nonpolar and nonporous surface, and the counterion-DNA complexes, which are absorbed easily on the surface. This disclosure provides a method to isolate target DNA fragments from a purified DNA pool. However, the application for purifying nucleic acids directly from blood is still not disclosed in this prior art because of possible column obstruction.

More discussion for the related method can be seen in the description of CA 2299119 in which a method for stabilizing and/or isolating nucleic acids is disclosed. The method described here uses at least two quaternary amines or cationic polymers with a phosphor group to precipitate and protect nucleic acids. However, complexes formed by strong cationic polymers and nucleic acids will increase the difficulty for purifying nucleic acids from cationic polymers.

Moreover, a novel composition for isolating and /or stabilizing nucleic acids from biological materials is disclosed in US2004014703. The object of the method of US2004014703 is to provide a composition for stabilizing RNA in the presence of tissue, blood, plasma, or serum. The composition comprises a cationic compound like quaternary amine for nucleic acids stabilization. There is no disclosure about isolating nucleic acids with primary amine, secondary amine or tertiary amine. Therefore, it is desirable to provide an improved method to mitigate and/or obviate the aforementioned problems.

### SUMMARY OF THE INVENTION

The object of the present invention is to provide a method to isolate nucleic acids from a biological sample with amino surfactants.

To achieve the object, the method of the present invention for isolating nucleic acids by forming an insoluble ionic complex between nucleic acids and a surfactant in a biological sample comprises a step of contacting the sample with an isolation composition consisted of amino surfactants of the formula (I):
R₁R₂R₃N(O)ₓ, (I)

wherein, R₁ and R₂ each independently is H, C1-C6 alkyl group, C6-C12 aryl group, or C6-C12 aralkyl group; R₃ is C1-C20 alkyl group, C6-C26 aryl group or C6-C26 aralkyl group; and x is an integer of 0 or 1.

The isolation composition of the present invention can be a liquid solution or a solid-state composition. The amino surfactants of the present invention can be any conventional amino surfactant. Preferably, the amino surfactant of the present invention is selected from the group consisting of dodecylamine, N-methyldodecylamine, N,N-dimethyldodecylamine, N,N-dimethyldodecylamine N oxide and 4-tetradecylaniline. The weight percentage of amino surfactants is not limited. Preferably, the weight percentage of amino surfactants in the solid-state composition is less than 90%, preferably, from 10% to 90%. When the amino surfactant and the isolation composition both represent as solutions, the concentration of the amino surfactants in the composition preferably ranges from 0.001% to 20%.

The method of the present invention can be performed without any presence of nonionic detergents or acids. However, the use of nonionic detergents, acids or the mixture thereof accompanied with specific amine surfactants achieves effectively results. Accordingly, the isolation composition with amino surfactants can selectively further comprises at least one nonionic detergent. The nonionic detergent can represents as a liquid detergent or a solid one in the isolation composition. The concentration of said nonionic detergent preferably ranges from 0.01% to 20% while the isolation composition is in a liquid state; the weight percentage of the detergent ranges from 0.01% to 40% while the isolation composition is in a solid state. The nonionic detergent of the present invention can be any conventional nonionic detergent; preferably, the nonionic detergent is polyoxyethylene sorbitan monolaurate. More preferably, the nonionic detergent is Tween 20 or Triton X-100, the most preferably, the nonionic detergent is Tween 20. The isolation composition with amino surfactants of the present invention can selectively further comprises at least one acid. The acid can be acid buffers or acid agents in a solid-state. The concentration of the acid buffer is less than 1 M. Preferably, the concentration of the acid buffer ranges from 0.01 to 0.5M. The acid can be any conventional one. More preferably, the acid is selected from a group consisting of maleic acid, tartaric acid, citric acid, and oxalic acid. The pH of the isolation composition of the present invention can be any value ranging from 1 to 14. Preferably, the pH ranges from 1 to 10.

The biological sample with nucleic acids used may be cell-free sample material, plasma, body fluids such as blood, serum, cells, leucocyte fractions, crusta phlogistica, sputum, urine, sperm, faeces, smears, aspirates, tissue samples of all kinds, such as biopsies, for example, parts of tissues and organs, food samples which contain free or bound nucleic acids or cells containing nucleic acids as envisaged according to the invention, such as organisms (single- or multi-cell organisms; insects, etc.), plants and parts of plants, bacteria, viruses, yeasts and other fungi, other eukaryotes and prokaryotes, etc.

The term “nucleic acids” for the purposes of the present invention denotes nucleic acids in the wider sense, and thus includes, for example, ribonucleic acids (RNA) and also deoxyribonucleic acids (DNA) in all lengths and configurations, such as double-stranded, single-stranded, circular and linear, branched, etc., and all possible subunits thereof, such as monomeric nucleotides oligomers, plasmids, viral and bacterial DNA and RNA, as well as genomic and non-genomic DNA and RNA from animal and plant cells or other eukaryotes, mRNA in processed and unprocessed form, tRNA, hn-RNA, rRNA, cDNA as well as all other conceivable nucleic acids. Preferably, the nucleic acids of the present invention are DNA or RNA.

Other objects, advantages, and novel features of the invention will become more apparent from the following detailed description when taken in conjunction with the accompanying drawings.

### BRIEF DESCRIPTION OF THE DRAWINGS

FIG. 1 is an electrophoresis result of purified nucleic acids with dodecylamine from whole blood.

FIG. 2 is an electrophoresis result of purified nucleic acids with N,N-dimethyldodecylamine from whole blood.

FIG. 3 shows electrophoresis results of purified nucleic acids with N,N-dimethyldodecylamine N oxide from whole blood.

### DETAILED DESCRIPTION OF THE PREFERRED EMBODIMENT

### Example 1

Purification of Total RNA From Whole Blood

Three different procedures were used to isolate total RNA from fresh whole blood. First of all, RNA was isolated using Red Blood Cell Lysis Buffer (Roche Diagnostics GmbH) and with a reagent (Trizol Reagent, Life Technologies) containing phenol according to the procedures described in the handbook as recommended by the vendor. Secondly, the PAXgene Blood RNA Validation Kit (QIAGEN GmbH) was used according to the supplier's handbook. Finally, the purification of total RNA was performed using primary amine surfactant as per the purification process described below.

First of all, a lysis buffer consisting of 1% (W/V) dodecylamine, 50 mM maleic acid, and 3% (V/V) of Tween 20 was prepared. Fresh human whole blood (333 μl) was mixed with 1 ml of the prepared lysis buffer, and then the mixture was rotated at a speed of 11 rpm for 20 minutes to ensure homogeneity. The mixture was centrifuged at 5000 xg for 10 minutes, and the supernatant was then discarded; the pellet was washed with 333 μl of double distilled water. Then the solution containing the pellet was centrifuged at 5000 xg for 10 minutes and the supernatant was discarded entirely.

147 μl of Buffer RLT (QIAGEN GmbH) was added and mixed with the sample thoroughly. Then 200 μl of 1-bromo-3-chloropropane was added and vortexed for 10 seconds. The sample was centrifuged at 10000 xg for 5 minutes and then the supernatant was transferred to a new tube. 90 μl of 100% ethanol was added and mixed by pipetting for three times, and then the solution was transferred to a RNeasy mini column (QIAGEN GmbH).

The sample was centrifuged at 13000 rpm for 30 seconds, then the flow-through was discarded; 350 μl of Buffer RW1 was added into the column, and the mixture was centrifuged again at 13000 rpm for another 30 seconds. The flow-through was then discarded. 10 μl of DNase I stock solution (QIAGEN GmbH) was added into 70 μl Buffer RDD. The DNase I incubation mixture was added directly into the RNeasy mini column, and was incubated at 20-30° C. for 15 minutes; 350 μl of Buffer RW1 was added into the column, then centrifuged at 13000 rpm for 30 seconds, and the flow-through was discarded.

500 μl of Buffer RPE (QIAGEN GmbH) was added into the column, then the sample was centrifuged at 13000 rpm for 30 seconds, and the flow-through was discarded. Another 500 μl of Buffer RPE was added into the RNeasy column. The column was centrifuged at 13000 rpm for 30 seconds, and then the flow-through was discarded. The empty column was centrifuged at 13000 rpm for 1 minute, and then the column was transferred to a clean 1.5 ml tube. 40 μl of RNase-free water was added into the column, and the column was centrifuged at 13000 rpm for 1 minute. Another aliquot of 40 μl of RNase-free water was added, and the column was centrifuged at 13000 rpm for 1 minute. 80 μl of eluted solution was transferred to a new 1.5 ml tube.

### Example 2

Purification of Genomic DNA in Whole Blood

Three different procedures were used to isolate genomic DNA from fresh whole blood for comparison. In the first procedures, DNA was isolated using Red Blood Cell Lysis Buffer (Roche Diagnostics GmbH) and with a reagent (Trizol Reagent, Life Technologies) containing phenol according to the procedures described in the handbook as recommended by the vendor. The second procedure used the QIAamp DNA Blood Kit (QIAGEN GmbH) according to the supplier's handbook. Finally, in the third procedure, purification of total DNA by using primary amine surfactant is described below.

333 μl of fresh whole blood was mixed with 1 ml of a solution consisting of 1% (w/v) of dodecylamine, 3% (v/v) Tween 20 and 50 mM maleic acid. To isolate the DNA, the complexes of primary amine surfactant and DNA were centrifuged at 5000 xg for 10 min. The pellet was dissolved in a mixture of 200 μl of buffer AL (QIAGEN GmbH) and 20 μl of Proteinase K. After being incubated at 55° C. for 10 min, the sample was mixed with 200 μl of ethanol, and then applied to a spin column containing a silica membrane. The sample mixture was passed through the membrane under centrifugation. The silica membrane was washed with 500 μl of buffer AW1 (QIAGEN GmbH), then with 500 μl of buffer AW2 (QIAGEN GmbH), and finally with 200 μl of distilled water to elute the DNA molecules.

### Example 3

Quality and Quantities of Nucleic Acids

To evaluate the concentrations of isolated nucleic acids, measurements by spectrophotometer in OD260/280 were taken. Referring to Table 1, a comparison table of the quality and quantities with 3 different isolation methods is shown. The three methods are: A. isolation with primary amine of the present invention, B. commercial kits of PAXgene Blood RNA Validation Kit or QIAamp DNA Blood Kit, and C. isolation with conventional method employing RBC lysis and TrizolReagent.

TABLE 1

| | RNA | | DNA | |
|---|---|---|---|---|
| | quantity | RNA | quantity | DNA |
| Isolation | (μg/ml | quality | (μg/ml | quality |
| method | blood) | (OD 260/280) | blood) | (OD 260/280) |
| A | 9.60 | 2.01 | 7.80 | 1.75 |
| B | 4.56 | 1.74 | 37.00 | 1.66 |
| C | 3.76 | 2.06 | 27.52 | 2.00 |

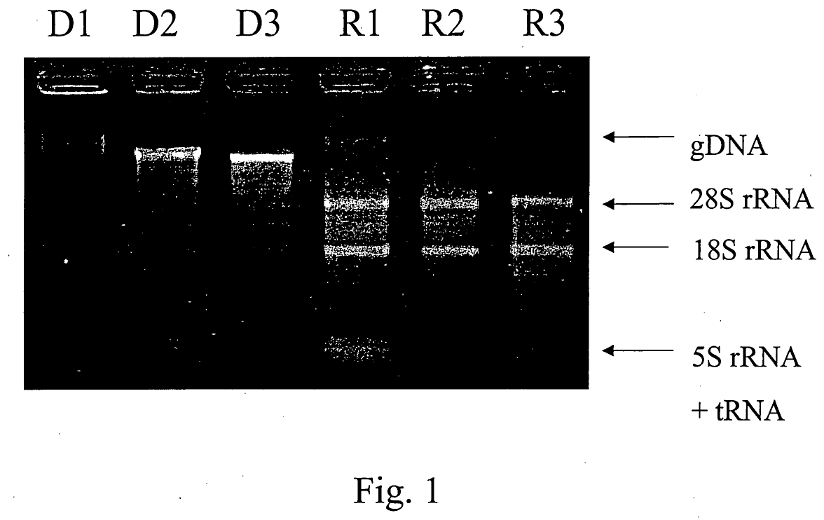

Based on the data of Table 1, isolation of RNA with primary amine of the present invention showed the highest quantity among these methods, and the quality was also in an ideal measurement range of 1.9 to 2.1. The data showed that the primary amine surfactant of present invention was also useful in DNA isolation. The resulted samples were further examined with the use of agarose gel electrophoresis. As shown in FIG. 1, lane D1 represented DNA isolation result by conventional method of RBC lysis and Trizol Reagent; lane D2 represented DNA isolation result by QIAamp DNA blood kit; and lane D3 represented DNA isolation result by primary amine surfactant of the present invention. Lane RI represented RNA isolation result by the conventional method of RBC lysis and Trizol Reagent; lane R2 represented RNA isolation result by PAX gene RNA blood kit; and lane R3 represented RNA isolation by primary amine surfactant of the present invention. The quantity of each loading lane was 100 ng of DNA or 200 ng of RNA. The results of lane D3 and lane R3 indicated that the quality of isolated nucleic acids by the method of example 1 was more efficient than the other two conventional methods. The results showed that the use of primary amine surfactant is capable of isolating DNA molecules with good quality. The quantity of the isolated RNA in lane R3 was about the same as the other two lanes. It is clearly shown from lane R3 that the method for isolating RNA with primary amine surfactant would obtain pure RNA molecules without genomic DNA contaminants.

### Example 4

Purification of Total RNA With Secondary Amine Surfactant

18 solutions of 1%, 2%, or 3% of surfactant N-methyldodecylamine and tartaric acid in various concentrations of 25, 50, 75, 100, 125, or 150 mM were prepared. 333 μl of fresh blood was mixed with 1 ml of each of the prepared surfactant solutions, and the sample mixture was rotated at a speed of 11 rpm for 20 minutes at room temperature.

The solution was centrifuged for 10 min at 5000 xg. The supernatant was then removed by decanting or pipetting. 666 μl of double-distilled water was then added to the pellet, and the pellet was resuspended thoroughly by vortexing. The sample was centrifuged for 10 min at 5000 xg. The entire supernatant was then removed and discarded. The pellet was resuspended thoroughly with 50 ρl of RNase free water by pipetting until no particle was visible. Then 100 μl of Buffer RLT (Qiagen, RNeasy mini kit) and 40 μl of Proteinase K were added and mixed uniformly by pipetting. The solution was incubated for 10 min at 55° C. in a shaker-incubator or water bath. 200 μl of 1-bromo-3-chloropropane was added into the sample and mixed by vortexing. The sample was then centrifuged for 5 min at 10000 xg. The supernatant was transferred into a new 1.5 ml tube.

90 μl of 100% ethanol was added into the sample, and mixed by vortexing. The mixture was then centrifuged briefly (less than 2 sec) at low speed to remove drops inside the tube lid.

The sample solutions were applied to a RNeasy mini column and centrifuged for 30 seconds at 13000 rpm. Then 350 μl of Buffer RW1 was added to the column and centrifuged for 1 min at 13000 rpm. The old processing tube containing flow-through was discarded.

DNase I stock solution 10 μl was added and mixed with 70 μl of Buffer RDD. The resulted 80 ul of DNase I incubation mixture was then transferred directly into the spin-column membrane, and the sample was placed at room temperature for 15 minutes. 350 μl of Buffer RW1 was applied to the column, and the spin-column was then centrifuged for 30 seconds at 13000 rpm. The old processing tube containing flow-through was discarded. 500 μl of Buffer RPE was applied to the column, and the column was centrifuged for 30 seconds at 13000 rpm. The old processing tube containing flow-through was discarded.

Another 500 μl of Buffer RPE was added into the column. The column was then centrifuged for 1 minute at 13000 rpm. The old processing tube containing flow-through was discarded. The column was centrifuged again for 2 minutes at 13000 rpm.

The column was transferred to a new 1.5 ml elution tube, then 40 μl of RNase-free water was pipetted directly onto the column membrane, and the sample tube was centrifuged for 1 minute at 13000 rpm. Another 40 μl of RNase-free water was applied into the column and centrifuged again, and the RNA product was flowed into elution tube after centrifugation.

The pellet size (%) of isolated RNA products acquired through the procedures illustrated above is shown in Table 2A.

TABLE 2A

| Tartaric | 25 | 50 | 75 | 100 | 125 | 150 |
|---|---|---|---|---|---|---|
| acid (mM) | mM | mM | mM | mM | mM | mM |
| 1% | 9 | 21 | 18 | 15 | 18 | 30 |
| 2% | 45 | 9 | 9 | 6 | 4.5 | 6 |
| 3% | 60 | 30 | 7.5 | 6 | 4.5 | 3 |

The pellet size was designated as the ratio of the pellet volume to the blood volume. Higher value of pallet size indicate that there are more impurities or contaminates carried in the pellet.

Table 2B shows the RNA yield (μg/ml blood) after RNA isolation followed the procedures above.

TABLE 2B

| Tartaric | 25 | 50 | 75 | 100 | 125 | 150 |
|---|---|---|---|---|---|---|
| acid (mM) | mM | mM | mM | mM | mM | mM |
| 1% | 1 | 4.26 | 3.82 | 2.74 | 2.86 | 1.25 |
| 2% | N/A | 3.61 | 3.42 | 2.24 | 2.57 | 2.25 |
| 3% | N/A | N/A | 3.69 | 3.36 | 2.9 | 2.24 |

Based on the data showed in Tables 2A and 2B, the formulation of 2% or 3% N-methyldodecylamine with 75-150 mM tartaric acid removed the highest amount of impurities or contaminates in the isolated RNA product, and the yields were satisfactory.

### Example 5

Purification of Total RNA With Tertiary Amine Surfactant

10 solutions of 1% or 3% of N, N-dimethyldodecylamine with tartaric acid in various concentrations of 50, 75, 100, 125, or 150 mM were prepared. 333 μl of fresh blood was then mixed with 1 ml of each of the prepared surfactant solutions, and the resulted sample mixture was rotated at room temperature at a speed of 11 rpm for 20 minutes to ensure homogeneity. The solution was then centrifuged for 10 min at 5000 xg. The supernatant was removed by decanting or pipetting. Then 666 μl of double distilled water was added to the pellet, and the pellet was resuspended thoroughly by vortexing. The mixture was centrifuged for 10 min at 5000 xg. The entire supernatant was then removed and discarded.

The pellet was resuspended thoroughly with 167 μl Buffer RLT (Qiagen, RNeasy mini kit) until no particle was visible. 200 μl of 1-bromo-3-chloropropane was added into the sample and mixed by vortexing. The sample was then centrifuged for 5 min at 10000 xg, and the supernatant was transferred into a new 1.5 ml tube.

90 μl of 100% ethanol was added into the sample and mixed by vortexing. The mixture was then centrifuged briefly (less than 2 seconds) at low speed to remove drops inside the tube lid.

The sample solutions were applied to RNeasy mini column, and centrifuged for 30 seconds at 13000 rpm. Then 350 μl of Buffer RW1 was added to the column, and centrifuged for 1 min at 13000 rpm. The old processing tube containing flow-through was discarded.

10 μl DNase I stock solution was added into 70 μl of Buffer RDD. The DNase I incubation mixture (80 μl) was then transferred directly onto the spin-column membrane, and the sample was placed at room temperature for 15 minutes. 350 μl of Buffer RW1 was applied to the column and centrifuged for 30 seconds at 13000 rpm. The old processing tube containing flow-through was discarded. 500 μl of Buffer RPE was applied to the column, and the column was centrifuged for 30 seconds at 13000 rpm. The old processing tube containing flow-through was then discarded.

Another 500 μl of Buffer RPE was added into the column. The column was then centrifuged for 1 minute at 13000 rpm, and then the old processing tube containing flow-through was discarded. The column was centrifuged again for 2 minutes at 13000 rpm.

The column was then transferred into a new 1.5 ml elution tube, then 40 μl of RNase-free water was applied directly into the column membrane, and the sample tube was centrifuged for 1 minute at 13000 rpm. Another 40 μl of RNase free water was applied into the column and centrifuged again.

The pellet size (%) and yields of isolated RNA products through the procedures illustrated above are shown in Table 3A and Table 3B, respectively.

TABLE 3A

| Tartaric | | | | | |
|---|---|---|---|---|---|
| acid (mM) | 50 mM | 75 mM | 100 mM | 125 mM | 150 mM |
| 1% | 7.5 | 6 | 10.5 | 7.5 | 9 |
| 3% | 2 | 1 | 1 | 1 | 1 |

TABLE 3B

| Tartaric | | | | | |
|---|---|---|---|---|---|
| acid (mM) | 50 mM | 75 mM | 100 mM | 125 mM | 150 mM |
| 1% | 5.16 | 5.07 | 3.07 | 2.5 | 1.94 |
| 3% | 1.75 | 5.24 | 3.05 | 2.4 | 2.84 |

Based on the data showed in Tables 3A and 3B, the formulation of 3% N,N-dimethyldodecylamine with 75 mM tartaric acid effectively removed the impurities or contaminates in the isolated RNA resulted in the highest quality of RNA products.

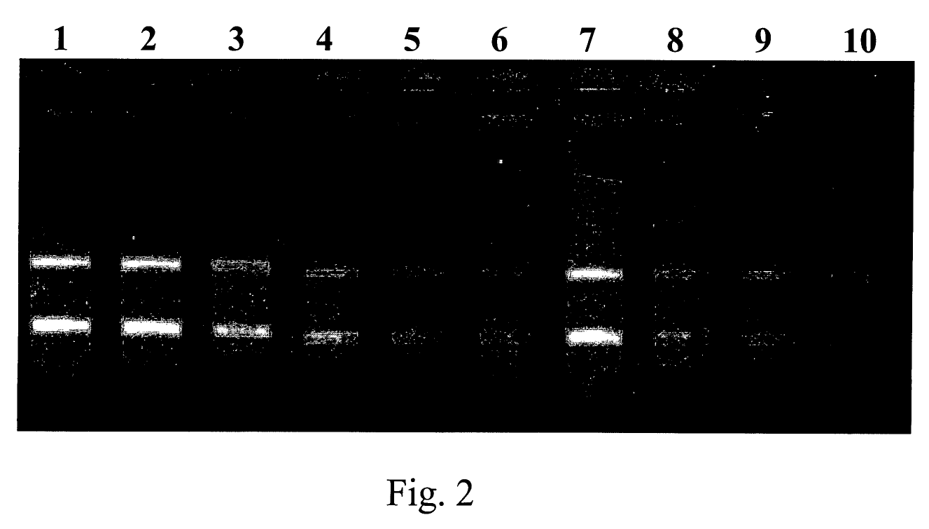

The isolation efficiency can be seen in the result of electrophoresis in FIG. 2, where lane 1 to land 5 were the lanes that 1% N,N-dimethyldodecylamine was used, and lane 6 to land 10 were the lanes that 3% N,N-dimethyldodecylamine was used. Lane 1 and 6, 2 and 7, 3 and 8, 4 and 9, 5 and 10 were formulated with 50 mM, 75 mM, 100 mM, 125 mM, and 150 mM tartaric acid, separately.

### Example 6

Purification of Total RNA With Tertiary Amine Oxide Surfactant

16 solutions were prepared, each containing 1% N, N-dimethyldodecylamine N oxide with various amount of maleic acid, citric acid, tartaric acid, or oxalic acid—in the concentrations of 25, 50, 75, or 100 mM. Then steps for isolating RNA from whole blood sample as described above in example 5 were followed.

Table 4 showed the RNA yields (μg/ml blood) after RNA isolation by following the above procedures.

TABLE 4

| RNA yield | 25 mM | 50 mM | 75 mM | 100 mM |
|---|---|---|---|---|
| Maleic acid | 4.32 | 3.12 | 3.84 | 2.40 |
| Citric acid | 3.84 | 3.12 | 4.56 | 3.36 |
| Tartaric acid | 2.64 | 4.08 | 3.60 | 3.84 |
| Oxalic acid | 5.28 | 4.56 | 3.36 | 3.60 |

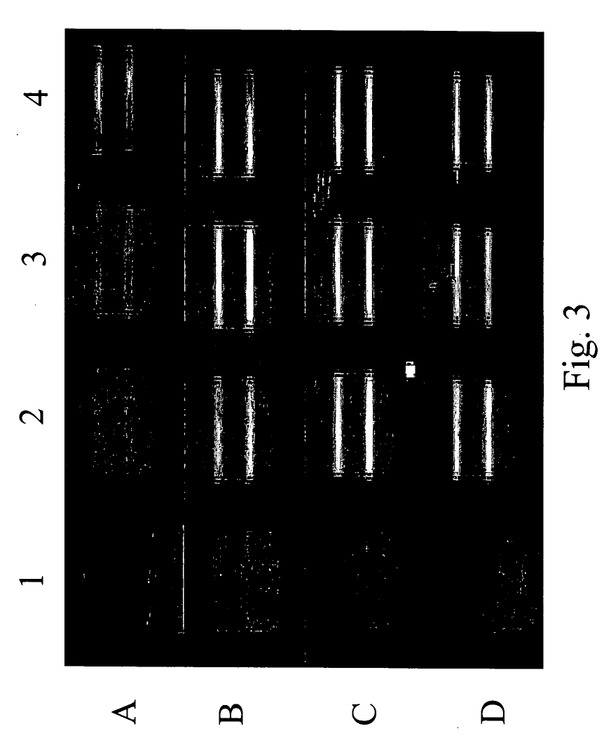

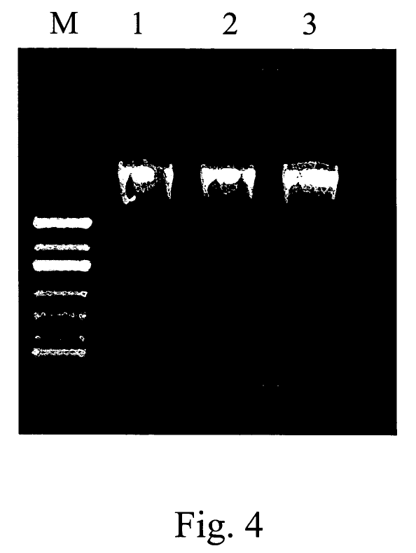

The isolation efficiency can be seen from the result of electrophoresis in FIG. 3, where part A was the result performed with maleic acid; part B was the result with citric acid; part C was the result with tartaric acid, and part D was the result with oxalic acid. Lane 1 was with a concentration of 25 mM, lane 2 was with 50 mM, lane 3 was with 5 mM, and lane 4 was with 100 mM.

According to the data in Table 4 and the electrophoresis result in FIG. 3, the RNA samples isolated with 1% N, N-dimethyldodecylamine N oxide and citric acid, tartaric acid or oxalic acid all showed good quality and acceptable quantities.

### Example 7

Purification of Genomic DNA in Whole Blood With 4-Tetradecylaniline

3 solutions of 1% 4-tetradecylaniline and Tween 20 in various concentrations of 3, 4, or 5% were prepared. 333 μl of fresh whole blood was mixed with 1 ml of each of the prepared surfactant solutions. The mixture was then centrifuged at 5000 xg for 10 min to precipitate the complexes of the DNA molecules and primary amine surfactants. The pellet was dissolved in 333 μl of buffer RLT (QIAGEN GmbH). Then 200 μl of 1-bromo-3-chloropropane was added into the sample and mixed by vortexing. The sample was then centrifuged for 5 min at 10000 xg.

The supernatant was transferred into a new 1.5 ml tube, then mixed with 160 μl of ethanol, and applied to a spin column containing a silica membrane. The sample was passed through the membrane under centrifugation. The silica membrane was washed with 700 μl of buffer RW1 (QIAGEN GmbH) once, then twice with 500 μl of buffer RPE (QIAGEN GmbH), and finally with 80 μl of distilled water to elute the DNA molecules.

The isolation efficiency can be seen from the result of electrophoresis in FIG. 4, wherein the 3 lanes are represented with different concentrations of Tween-20. Lane 1 was with a concentration of 3%, Lane 2 was with a concentration of 4% and Lane 3 with was a concentration of 5%. According to the result, the DNA samples isolated with 4-tetradecylaniline showed good quality and acceptable quantity. In addition, an intact genomic DNA can be extracted.

Although the present invention has been explained in relation to its preferred embodiment, it is to be understood that many other possible modifications and variations can be made without departing from the spirit and scope of the invention as hereinafter claimed.

What is claimed is:

1. A method for isolating nucleic acids by forming an insoluble ionic complex between nucleic acids and a surfactant in a biological sample, comprising a step of contacting the biological sample with an isolation composition comprising amino surfactants of the formula (I):
R₁R₂R₃N(O)ₓ, (I),
wherein, R₁ and R₂ each independently is H, C1 -C6 alkyl group, C6-C12 aryl group, or C6-C12 aralkyl group; R₃ is C1-C20 alkyl group, C6-C26 aryl group or C6-C26 aralkyl group; and x is an integer of 0 or 1.

2. The method according to claim 1, wherein x is 1, R₁ and R₂ each independently is C1-C6 alkyl, and R₃ is C1-C20 alkyl.

3. The method according to claim 1, wherein x is 0.

4. The method according to claim 1, wherein said amino surfactants are selected from the group consisting of dodecylamine, N-methyldodecylamine, N,N-dimethyldodecylamine, N,N-dimethyldodecylamine N oxide and 4-tetradecylaniline.

5. The method according to claim 1, wherein the isolation composition is an aqueous medium.

6. The method according to claim 5, wherein the concentration of said amino surfactants in the composition ranges from 0.001% to 20%.

7. The method according to claim 1, wherein the isolation composition is in a solid state.

8. The method according to claim 7, wherein the weight percentage of said amino surfactants in the composition ranges from 10% to 90%

9. The method according to claim 1, wherein said isolation composition further comprises nonionic detergents, acid salts, or the mixture thereof.

10. The method according to claim 9, wherein said nonionic detergent is polyoxyethylene sorbitan monolaurate.

11. The method according to claim 9, wherein said nonionic detergent is Tween 20.

12. The method according to claim 9, wherein the isolation composition is in a liquid state, and the concentration of said nonionic detergents in the composition ranges from 0.01% to 20%.

13. The method according to claim 9, wherein the isolation composition is in a solid state, and the percentage weight of said nonionic detergents in the composition ranges from 0.01% to 40%.

14. The method according to claim 9, wherein said acid salt is selected from a group consisting of maleic acid, tartaric acid, citric acid, oxalic acid, carboxylic acids and mineral acid.

15. The method according to claim 9, wherein the total concentration of said acid salts in the isolation composition ranges from 0.01 M to 1M.

16. The method according to claim 1, wherein the pH of said isolation composition ranges from 1 to 10.

17. The method according to claim 1, wherein said biological sample is selected from the group consisting of whole blood, plasma, serum, urine, tissue and cells.

18. The method according to claim 1, wherein said nucleic acids are DNA and RNA.
